(19) United States
(12) Patent Application Publication
(10) Pub. No.: US 2016/0251682 A1
(43) Pub. Date: Sep. 1, 2016

(54) FRACTIONATION OF LIGNOCELLULOSIC BIOMASS FOR CELLULOSIC ETHANOL AND CHEMICAL PRODUCTION

(71) Applicant: GreenField Specialty Alcohols Inc., Toronto (CA)

(72) Inventors: Frank A. DOTTORI, Temiscaming (CA); Robert Ashley Cooper BENSON, North Bay (CA); Regis-Olivier BENECH, Chatham (CA)

(21) Appl. No.: 15/150,946

(22) Filed: May 10, 2016

Related U.S. Application Data
(63) Continuation-in-part of application No. 13/551,087, filed on Jul. 17, 2012, now abandoned.
Application No. 13/551,087 is a continuation-in-part of application No. 13/460,207, filed on Apr. 30, 2012, now abandoned.
Application No. 13/460,207 is a continuation of application No. 12/766,599, filed on Apr. 23, 2010, now abandoned.
(60) Provisional application No. 61/172,057, filed on Apr. 23, 2009.
(60) Provisional application No. 61/171,997, filed on Apr. 23, 2009.

(51) Int. Cl. C12P 7/14; D21C 3/22

(52) U.S. Cl. CPC C12P 7/14; D21C 3/22; C12P 2201/00

(57) ABSTRACT

A process is defined for the continuous steam pretreatment and fractionation of low lignin lignocellulosic biomass to produce a concentrated cellulose solid stream that is sensitive to enzymatic hydrolysis. Valuable chemicals are recovered by fractionating the liquid and vapor stream composed of hydrolysis and degradation products of the hemicellulose. Cellulosic derived glucose is produced for fermentation to biofuels. A xylo-oligosaccharides rich liquids fraction is recovered that can be converted to value added products including ethanol.

### CROSS REFERENCE TO RELATED APPLICATIONS

This application is a continuation-in-part of U.S. patent application Ser. No. 13/551,087 filed Jul. 17, 2012, which is a continuation-in-part of U.S. patent application Ser. No. 13/460,207 filed Apr. 30, 2012, which is a continuation of U.S. patent application Ser. No. 12/766,599 filed Apr. 23, 2010, which claims the benefit of priority of U.S. Provisional Patent Application No. 61/172,057 filed Apr. 23, 2009, of U.S. Provisional Application No. 61/171,997 filed Apr. 23, 2009, all of which are hereby incorporated by reference.

### FIELD OF THE INVENTION

The present invention generally relates to the production of ethanol from lignocellulosic biomass and in particular to a process for extracting cellulose and hemicellulose fractions from low lignin containing biomass.

### BACKGROUND OF THE INVENTION

Concerns over high oil prices, security of supply and global warming have raised the demand for renewable energy. Renewable energy is energy produced from plant derived biomass. Renewable energy applications such as fuel ethanol are seen as a valuable contribution to the reduction in fossil fuel consumption. Public policies have supported the creation of a fuel ethanol industry largely based on the use of corn as a feedstock. The production of fuel ethanol helps to stabilize farm income and reduces farm subsidies. However, as demand increases for fuel ethanol, additional feedstocks such as lignocellulosic biomass are under consideration.

Fuel ethanol is created by the fermentation of starch derived sugars. The ethanol is distilled and dehydrated to create a high-octane, water-free gasoline substitute. Fuel ethanol is blended with gasoline to produce a hybrid fuel, which has environmental advantages when compared to gasoline alone, and can be used in gasoline-powered vehicles manufactured since the 1980's. Most gasoline-powered vehicles can run on a blend consisting of gasoline and up to 10 percent ethanol, known as “E-10”.

While corn is currently the major raw material for producing ethanol in North America, it is already apparent that large-scale use of ethanol for fuel will require new technologies that will allow the industry to expand its feedstock options to include cellulose.

Cellulosic ethanol is manufactured from lignocellulosic biomass. Lignocellulosic biomass may be grouped into four main categories: (1) wood residues (including sawmill and paper mill discards), (2) municipal paper waste, (3) agricultural wastes (including corn stover, corn cobs and sugarcane bagasse), and (4) dedicated energy crops which are mostly composed of fast growing tall, woody grasses such as switch grass and Miscanthus.

Lignocellulosic biomass is composed of three primary polymers that make up plant cell walls: Cellulose, hemicellulose and lignin. Cellulose is a polymer of D-glucose. Hemicellulose contains two different polymers i.e. xylan, a polymer of xylose and glucomannan, a polymer of glucose and mannose. Lignin is a polymer of guaiacylpropane- and syringylpropane units.

In lignocellulosic biomass, cellulose fibers are locked into a rigid structure of hemicellulose and lignin. Lignin and hemicelluloses form chemically linked complexes that bind water soluble hemicelluloses into a three dimensional array, cemented together by lignin. The cellulose is present as microfibrils. The lignin covers the cellulose microfibrils and protects them from enzymatic and chemical degradation. These polymers provide plant cell walls with strength, but also provide resistance to degradation, which makes lignocellulosic biomass a challenge to use as a substrate for biofuel production. Relatively small variations in the content or organization of these polymers from biomass to biomass generate significant differences in the results of conventional biomass treatment processes. A large number of different processes are therefore used for cellulosic ethanol production from lignocellulosic biomass.

Cellulose, or poly-1-4-glucan, is a linear polysaccharide polymer of glucose made of cellobiose units. The cellulose chains are packed by hydrogen bonds into microfibrils. These fibrils are attached to each other by the hemicelluloses and are covered by the lignin.

Hemicellulose is a physical barrier which surrounds the cellulose fibers and protects cellulose against degradation. Moreover, hemicellulose also has a chemical protection effect, since there is evidence that hemicellulose, containing xylose polymers (xylan), as well as its hydrolysis breakdown products inhibit the activity of cellulolytic enzymes. This chemical inhibition has a negative effect on cellulose to glucose conversion rates. Thus, for the production of fermentable sugars and ethanol from cellulose, it is desirable to generate a highly reactive cellulose with a low xylan content for the enzymatic hydrolysis to the fermentable sugars. Moreover, fermentation of glucose derived from lignocellulosic biomass is generally inhibited by the xylan breakdown products of xylooligosaccharides and xylose simply because most of the C6 sugar fermenting organisms are unable to process C5 sugars with the same range of performance.

Lignin is a very complex molecule constructed of phenylpropane units linked in a three dimensional structure which is particularly difficult to biodegrade. Lignin is the most recalcitrant component of the plant cell wall. There are chemical bonds between lignin, hemicellulose and cellulose polymers. There is evidence that the higher the proportion of lignin, the higher the resistance to chemical and biological hydrolysis. Lignin and some soluble lignin derivatives have been found to inhibit enzymatic hydrolysis and fermentation processes. Thus, it is desirable to generate a highly reactive cellulose which is low in xylan content and is low in lignin content.

Published work on the various processes for the production of fermentable sugars from cellulosic biomass shows the existence of an inverse relationship between lignin content and the efficiency of enzymatic hydrolysis of sugar based polymers. Lignocellulosic microfibrils are associated in the form of macrofibrils. This complicated structure and the presence of lignin provide plant cell walls with strength and resistance to degradation, which also makes these materials a challenge to use as substrates for the production of biofuel and bioproducts. Thus, pretreatment is necessary to produce highly reactive cellulose reacting well with catalysts such as enzymes.

The products obtainable with pretreatment include purified cellulose, xylose and lignin-free xylo-oligosaccharides are valuable for many purposes. Specifically, reactive cellulose extracted from biomass with low lignin content may be easily hydrolyzed to fermentable sugar monomers and then fermented to ethanol and other biofuels. However, hemicellulose hydrolysis generates not only xylose and xylo-oligosaccharides, but also inhibitory degradation products which must be separated from the xylo-oligosaccharides stream to make the xylo-oligosaccharides extracted from the hemicellulose fraction valuable and useful in the preparation of value added products, for example prebiotic substances for food and pharmaceutical applications.

It is generally accepted in the field that the best pretreatment method and conditions will depend greatly on the type of lignocellulosic starting material used. Pretreatment configuration and operating conditions must be adjusted with respect to the content or organization of the above discussed lignocellulosic polymers in the starting material, if one is to attain optimal conversion of cellulose to fermentable sugars. The cellulose-to-lignin ratio is the main factor. However, other parameters which play a significant role are the content of hemicellulose, degree of acetylation of hemicellulose, cellulose-accessible surface area, degree of polymerization and crystallinity. For example, the lignin content of corncobs and certain hybrids of Miscanthus for example, is similarly low i.e. 5% to 10%. Yet, their contents of cellulose and hemicellulose are very different with the ratios of cellulose:lignin:hemicellulose for Corncobs and Miscanthus being 8:1:7 and 5:1:2, respectively. Thus, despite their similar lignin contents, Corncobs and Miscanthus are generally subjected to significantly different pre-treatment conditions. It is this variability from biomass to biomass in the required pre-treatment conditions, which makes it very difficult to develop a single, efficient process for use with different biomasses, or more importantly, biomass mixtures. A single process for the efficient treatment of biomass mixtures would of course be desirable, since that would obviate the need to supply only a specific biomass or an assorted biomass stream for ethanol production.

It is generally thought that an effective pretreatment should: (a) produce reactive cellulosic fiber for enzymatic attack, (b) minimize destruction of cellulose and hemicelluloses, and (c) minimize the formation of inhibitors for hydrolytic enzymes and fermenting microorganisms.

Several methods have been investigated for the pretreatment of lignocellulosic materials to produce reactive cellulose. These methods are classified into physical pretreatments, biological pretreatments and physicochemical pretreatments.

The prior art teaches that physical and biological pretreatments are not suitable for industrial applications. Physical methods such as milling, irradiation and extrusion are highly energy demanding and produce low grade cellulose. Also, the rates of known biological treatments are very low.

Pretreatments that combine both chemical and physical processes are referred to as physicochemical processes. These methods are among the most effective and include the most promising processes for industrial applications. Hemicellulose hydrolysis and lignin removal are often nearly complete. Increase in cellulose surface area, decrease in cellulose degree of polymerization and crystallinity greatly increase overall cellulose reactivity. Treatment rates are usually rapid. These pretreatment methods usually employ hydrolytic techniques using acids (hemicellulose hydrolysis) and alkalis for lignin removal.

The steam explosion process is well documented. Batch and continuous processes have been tested at laboratory and pilot scale by several research groups and companies. In steam explosion pretreatment, biomass is treated at high pressure, and high temperatures under acidic conditions i.e. 160° C. to 260° C. for 1 min to 20 min, at pH values <pH 4.0. The pressure of the pretreated biomass is suddenly reduced, which makes the materials undergo an explosive decompression leading to defibrization of the lignocellulosic fibers.

Steam explosion pretreatment is not very effective in dissolving lignin, but it does disrupt the lignin structure and increases the cellulose susceptibility to enzymatic hydrolysis. Steam explosion pretreatment generally results in extensive hemicellulose breakdown and, to a certain extent, to the degradation of xylose and glucose.

Steam explosion pretreatment has been successfully applied to a wide range of lignocellulosic biomasses. Acetic acid, sulfuric acid or sulfur dioxide are the most commonly used hydrolysis catalysts.

In one variant of the steam explosion pretreatment process, the autohydrolysis process, no acid catalyst is added to the biomass, as long as pH values below 4.0 are achieved in the pretreatment process. This is made possible by the release of acetic acid during the breakdown of acetylated hemicellulose by high pressure steam during the biomass cooking stage. However, the degree of hemicellulose acetylation, also referred to as acetyl group content, is highly variable among different biomasses, which again makes it difficult to develop a single set of process conditions useful for different biomasses. The hemicellulose content of corncobs is high, much of the hemicellulose in corncobs is acetylated. It is therefore relatively easy to achieve a pH value below 4.0 in the pretreated biomass, which means the breakdown and solubilization of the hemicellulose for release of the cellulose is achieved without acid addition. Thus, one could theorize that other biomasses could be treated equally well if acetic acid were added in an amount sufficient to achieve a pH of 4.0 in the treated biomass.

That however does not hold true, for example, for the pretreatment of Miscanthus, which does not have a high degree of acetylation. To achieve a degree of hemicellulose hydrolysis similar to that of the autohydrolysis pretreatment process for highly acetylated biomass, such as corncobs, Miscanthus requires the addition of sufficient acid prior to the steam heating process to reach a pH of 2.0.

Consequently, although the presence of acetic acid in the biomass reduces the need for acid catalysts, most known steam explosion pretreatment processes include the use of an acid catalyst. Yet, mineral acids, acetic acid and other carboxylic acids are all powerful inhibitors of the cellulose hydrolysis process as well as the downstream glucose fermentation process. Mineral and carboxylic acids added during pretreatment often remain in the pretreated biomass and carry through to the hydrolysis and fermentation steps, decreasing the efficiency of the overall ethanol production process. In addition, although acids may be used to catalyze the hydrolysis of hemicellulose, they also lead to unwanted decomposition of the sugars released in the process, thereby reducing the value of the decomposition products obtained during pretreatment, since the sugar breakdown products also have an inhibitory effect on downstream hydrolysis and fermentation processes and must be removed. Consequently the cellulose must be cleaned prior to the cellulose hydrolysis step to remove residual acids and inhibitory components generated by the action of the acid catalysts, which renders the overall process inefficient.

WO2007/009463 (Holm et al.) teaches a method for the conversion of cellulosic material to ethanol. Holm et al. teach a process for fractionation of lignocellulosic biomass into a fiber fraction and a liquid fraction. The process includes hydrothermal pretreatment at low severity to reduce capital and operational cost, to retain the majority of the hemicellulose and lignin in the fiber fraction and to minimize the formation of inhibitors. The process conditions are chosen so that only a minor part of the hemicellulose and hemicellulose hydrolysis products are transferred into the liquid fraction. Inhibitors created during hydrothermal pretreatment are concentrated in the liquid fraction. Chemical detoxification of the liquid fraction with NH₃ is taught. After hydrolysis of the cellulose in the fiber fraction, the C5 and C6 sugars in the reaction mixture can be co-fermented for ethanol production. Fractionation of the biomass into a solids fraction including mainly cellulose and a liquids fraction including mainly xylo-oligosaccharides without chemical detoxification is not possible with the method of Holm et al.

US2011/0065785 (Larson) teaches methods for bioethanol fermentation from lignocellulosic biomass and methods of processing lignocellulosic biomass. The biomass is pretreated at conditions resulting in a severity index of at least 3 and the fiber fraction of the pretreated biomass is then processed in a manner to minimize the effect of inhibitors in the fiber fraction on downstream fermentation. Dilution with water is used for reducing the effect of the inhibitors contained in the pretreated biomass. Higher levels of dilution are taught for biomass pretreated at higher severities. Reduction of the inhibitors content in the fiber fraction through other methods or during the pretreatment step is not disclosed. Methods for fractionation of the biomass into solids and liquids other than pressing with or without washing are not disclosed.

### SUMMARY OF THE INVENTION

It is now an object of the present invention to provide a process which overcomes at least one of the above disadvantages.

The inventors have now surprisingly discovered, that the most important treatment conditions during pretreatment for the achievement of autohydrolysis of acetyl group containing biomasses are neither the pH of the treated biomass, which means the amount of added acid catalyst, nor the type of acid catalyst used, but rather the severity (temperature/pressure and residence time) of the steam treatment. Moreover, the inventors have surprisingly discovered, that any biomass with a lignin content below 12% by weight on a dry matter basis and an acetyl group content of 3-6% by weight on a dry matter basis can be subjected to autohydrolysis without the addition of any acid catalyst not inherent in the starting biomass, thereby minimizing the amount of residual acid in the pretreated biomass. In particular, the inventors have surprisingly discovered that all those different types of biomass can be successfully treated to achieve highly digestible cellulose by using exactly the same steam pretreatment conditions. The inventors surprisingly discovered steam pretreatment conditions which will result in a cellulose of equal digestibility for biomasses of such diverse content as corncobs (8:1:7, cellulose:lignin:hemicellulose) and bagasse (1.8:1:1.3), without the addition of any acid catalyst and without controlling the amount of acetic acid released in the pretreatment step as long as the lignin content of the biomass is below 12% and the biomass has an acetyl group content of 3-6% by weight on a dry matter basis.

The inventors have further discovered that for these types of biomass (acetyl group content of 3-6%) autohydrolysis of the hemicellulose fraction can be advantageously used not only to generate a cellulose rich solids fraction, but also a xylo-oligosaccharides rich liquid fraction, since autohydrolysis of this type of biomass at an elevated severity of about 4 results in elevated hemicellulose hydrolysis. However, autohydrolysis at this severity also results in an elevated level of hemicellulose breakdown and degradation products with a detrimental effect on the catalytic activities of cellulolytic enzymes, which outweighs the benefits of increased hemicellulose breakdown and cellulose release. To address this problem, the inventors have further developed a sequence of purging, liquid extraction and purification steps carried out in conjunction with the steam pretreatment at a severity of about 4, which steps allow the fractionation of this type of biomass into a cellulose rich solids fraction, a xylo-oligosaccharides rich liquid fraction and an inhibitors containing vapor fraction. That means the process of the invention provides not only a highly digestible cellulose stream with low inhibitors content, but also a liquid xylo-oligosaccharides stream with low inhibitors content, without the need for chemical detoxification, thereby maximizing simultaneous C6 and C5 extraction from the biomass and improving the economics of the process. As is apparent from the above discussion of known approaches, improving the overall ethanol yield and reducing enzyme usage or hydrolysis time are generally linked to increased operating costs. The increased costs may outweigh the value of the increased ethanol yield, rendering existing methods economically unacceptable.

The inventors have not only discovered a single pretreatment process useful for a range of different biomasses, but an autohydrolysis process that generates cellulose having the same digestibility as cellulose obtained from corncobs, the biomass previously believed to have the best acetyl group content for autohydrolysis.

When this process is then combined with the steps of purging impurities during steam pretreatment and liquid extraction of inhibitory substances resulting from the hemicellulose autohydrolysis prior to cellulose hydrolysis, an economical process to convert low lignin lignocellulosic biomasses to fermentable sugar is achieved, due to the added commercial value of a separately obtained xylo-oligosaccharides rich liquid fraction.

In addition, the value of the byproduct streams from the process is maximized by separately capturing an inhibitors rich vapor fraction. As an example, xylo-oligosaccharides, (non digestible sugar oligomers made up of xylose units), have beneficial health properties; particularly their prebiotic activity. This makes them good candidates as high value added bioproducts.

In summary, a process is described for the continuous steam explosion pretreatment of biomass with a lignin content below 12% and an acetyl group content of 3-6% weight/weight on a dry matter basis by autohydrolysis of the biomass at a severity index of about 4, without the addition of any acid catalyst and without controlling the amount of acetic acid released in the pretreatment step or the pH of the treated biomass, which process results in a cellulose rich solids fraction, a xylo-oligosaccharides rich liquid fraction and an inhibitors rich vapor fraction.

Preferably, the pretreated biomass is extracted prior to cellulose hydrolysis, which means either while still under pressure prior to exiting the pretreatment reactor or after exiting the reactor, or both. Extraction refers in general to a single or multiple step process of removing liquid portions from the fibers with or without addition or utilization of an eluent, (the diluting step). Minimal water is preferably used as an eluent to remove water soluble hemicellulose and cellulose degradation products generated during autohydrolysis, such as, xylose, xylo-oligosaccharides, furans, fatty acids, sterols, ester, ethers and acetic acid. The extraction can be enhanced by use of a mechanical compressing device such as a modular screw device. The eluent can be recycled to increase the economy of its use or used for example in the known process of counter current washing as an example. Liquefied components in the steam treated lignocellulosic biomass and the dissolved components are subsequently removed from the fibrous solids. Generally this removes most of the dissolved compounds, the wash water, primarily consisting of hemicellulose hydrolysis and degradation products that are inhibitory to downstream hydrolysis and fermentation steps.

The extracting system preferably uses a device that employs a mechanical pressing or other means to separate solids from liquid or air from solids. This can be accomplished under pressure as described above and/or under atmospheric pressure accomplished with several different types of machines that vary and the detail of which is not essential to this invention.

The extract stream containing the xylo-oligosaccharide fraction is collected and preferably concentrated to the desired dryness for further applications. A final refining step may be required for producing xylo-oligosaccharides with a degree of purity suitable for pharmaceuticals, food and feed, and agricultural applications. Vacuum evaporation can be applied in order to increase the concentration and simultaneously remove volatile compounds such as acetic acid and flavors or their precursors.

The biomass is preferably chopped or ground and preheated with live steam at atmospheric pressure prior to the pretreatment step. Air is preferably removed from the biomass by pressing. Liquefied inhibiting extracts can be removed at this time. As mentioned above, no acid for catalyzing the breakdown/hydrolysis of the hemicellulose is added. However, the biomass is cooked with steam at elevated temperatures and pressures for a preselected amount of time to achieve a severity index of about 4, which means 3.9 to 4.1. During the pretreatment purging of condensate and venting of volatiles is preferably carried out continuously.

The pressurized activated cellulose is preferably flashed into a cyclone by rapidly releasing the pressure to ensure an explosive decompression of the pretreated biomass into fibrous solids and vapors. This opens up the fibers to increase accessibility for the enzymes. Purified cellulose with a low level of residual hemicellulose can be sent to the hydrolysis and fermentation stages.

### BRIEF DESCRIPTION OF THE DRAWINGS

Other objects and advantages of the invention will become apparent upon reading the detailed description and upon referring to the drawings in which:

FIG. 1 shows a process diagram of the continuous pretreatment unit proposed in the example.

FIG. 2 shows the total percentage recovery of cellulose and hemicellulose produced during the fractionation of corncobs.

FIG. 3 illustrates the susceptibility of pretreated corncob cellulose to enzymatic hydrolysis i.e. cellulose to glucose conversion.

FIG. 4 shows hydrolysis and fermentation results using pretreated corncobs produced at pilot scale (2.5 metric tons, 17% consistency).

FIG. 5 shows the total percentage recovery of cellulose and hemicellulose produced during high pressure fractionation of corncobs.

FIG. 6 shows the total percentage recovery of cellulose and hemicellulose produced during low pressure fractionation of corncobs.

FIG. 7 shows hydrolysis and fermentation results using pretreated corncobs produced at pilot scale and low pressure.

FIG. 8 shows the total percentage recovery of cellulose and hemicellulose in solid and liquid fractions produced over the fractionation of Bagasse.

FIG. 9 shows hydrolysis and fermentation results using pretreated bagasse produced at pilot scale and high pressure;

FIG. 10 illustrates the susceptibility of pretreated cellulose from concob (Example 3) to enzymatic hydrolysis (cellulose to glucose conversion) and fermentability of hydrolyzed cellulose (glucose to ethanol conversion);

FIG. 11 illustrates the susceptibility of pretreated cellulose from bagasse (Example 4) to enzymatic hydrolysis (cellulose to glucose conversion) and fermentability of hydrolyzed cellulose (glucose to ethanol conversion); and

FIG. 12 illustrates the relative proportion of the solids, solubles and volatiles streams obtained by hemicellulose autohydrolysis during steam pretreatment of low lignin biomass at different severity indexes.

### DETAILED DESCRIPTION OF THE PREFERRED EMBODIMENTS

Before explaining the present invention in detail, it is to be understood that the invention is not limited to the preferred embodiments contained herein. The invention is capable of other embodiments and of being practiced or carried out in a variety of ways. It is to be understood that the phraseology and terminology employed herein are for the purpose of description and not of limitation.

The abbreviations used in the figures have the following meaning:

° C., temperature in degree Celsius

ms, millisecond

DM, Dry matter

SI, Severity Index

t_{90%}, Time to reach 90% of maximum theoretical cellulose to glucose conversion.

### Pretreatment of Lignocellulosic Biomass

This invention is a new process for fractionating lignocellulosic biomass with a lignin content below 12% by weight on a dry matter basis and an acetyl group content of 3-6% by weight on a dry matter basis, in particular a process for fractionating the lignocellulosic biomass into two main commercially valuable components, a cellulose-rich solids fraction and a xylo-oligosaccharides-rich liquids fraction (solution). The cellulose-rich component is valuable for many purposes, since it can be more easily hydrolyzed to glucose and in turn more easily fermented to ethanol or other biofuels than in previous processes.

A preferred aspect of the invention is a continuous process for the pretreatment of these types of lignocellulosic biomass solely by autohydrolysis, in the absence of any acid catalyst, thereby minimizing the amount of residual acid in the pretreated biomass. In particular, all those types of biomass are treated in accordance with the invention by using exactly the same steam pretreatment conditions to achieve highly digestible cellulose. This not only makes it possible to use the same process equipment for different types of biomass, thereby significantly lowering the capital cost for processing plants intended to treat different biomasses, but also allows for the treatment of a mixture of different biomasses, thereby providing much wider access to a larger amount of biomass sources.

The inventors discovered that using steam pretreatment without the addition of any acid catalyst and without controlling the amount of acetic acid released in the pretreatment step, when carried out at autohydrolysis conditions, will result in a cellulose of equal digestibility for biomasses of such diverse content as corncobs (8:1:7, cellulose:lignin:hemicellulose) and bagasse (1.8:1:1.3), as long as the lignin content of the biomass is below 12% and the biomass has an acetyl group content of 3-6% by weight on a dry matter basis, the latter being important for a successful operation at autohydrolysis conditions.

The intention of steam explosion pretreatment of lignocellulosic biomass is generally to create a solids fraction with easily digestible cellulose. For the generation of ethanol from lignocellulosic biomass, the primary goal is to maximize the cellulose recovery and accessibility. Separate recovery of the hydrolysis products of the hemicellulose portion of the biomass (mainly xylose, xylo-oligosaccharides and degradation products) is possible, but generally of little interest, since the hemicellulose hydrolysis products recovered include sugar degradation products that are inhibitory to fermentation to ethanol, or interfere with upgrading of the xylo-oligosaccharides into valuable downstream products. In existing processes for the production of ethanol from lignocellulosic biomass the hemicellulose breakdown and degradation products are often removed together during pretreatment to reduce their inhibitory effect on downstream cellulose hydrolysis and glucose fermentation. Although purified xylo-oligosaccharides can be of value as starting compounds in chemical and pharmaceutical products, the cost of treating the recovered stream of hemicellulose breakdown and degradation products to obtain a purified xylo-oligosaccharides stream render this option uneconomical. Thus, in other known processes for the production of ethanol from lignocellulosic biomass the hemicellulose breakdown and degradation products are first removed together during pretreatment and later combined with the cellulose hydrolysis products for co-fermentation with the cellulose hydrolysis products. Although the ethanol yield from fermentation of the hemicellulose hydrolysis products is only a fraction of that from the cellulose hydrolysis products, co-fermentation results in a better overall economic result than upgrading the hemicellulose hydrolysis and degradation products into starting compounds for chemical and pharmaceutical products.

The inventors have now developed a continuous process for fractionation of lignocellulosic biomass which allows for fractionation of the products obtained from autohydrolysis of lignocellulosic biomass not only into digestible cellulose and hemicellulose hydrolysis and breakdown products, but into a cellulose-rich solids fraction, a xylo-oligosaccharides-rich liquid fraction and a vapor fraction containing inhibitors to fermentation.

During testing of different steam explosion pretreatment process options, the inventors discovered, first, that autohydrolysis be carried out using the same general treatment conditions for lignocellulosic biomass with a lignin content of less than 12% as long as the acetyl-group content is 3-6% by weight on a dry matter basis and, second, that fractionation of the hemicellulose in the biomass can be improved in such a manner to render the creation of purified xylo-oligosaccharides economical.

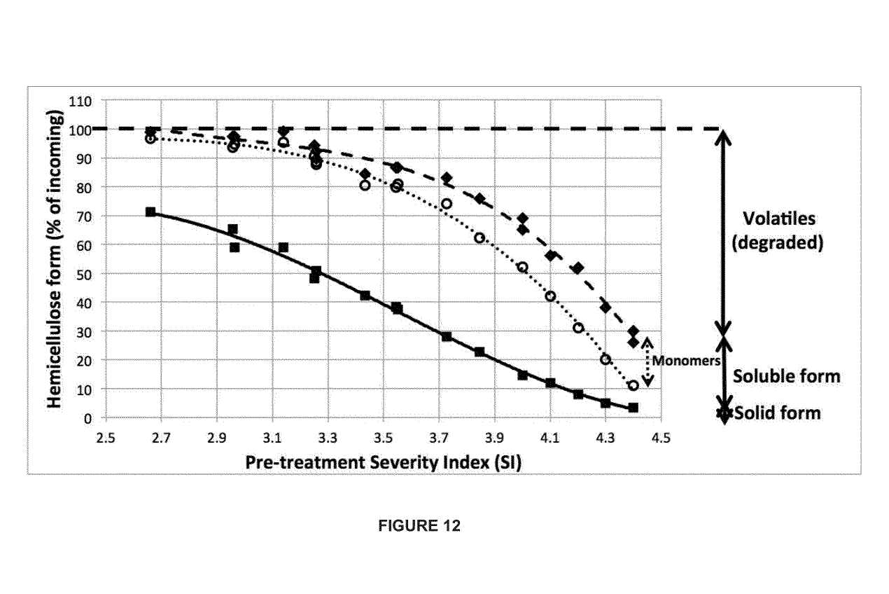

The inventors discovered that the hemicellulose originally in the lignocellulosic biomass in solid form decomposed during steam pretreatment into a solids fraction, a solubles fraction and a volatiles fraction, the solubles fraction including a monomeric fraction (mainly xylose monomers) and an oligomeric fraction (mainly xylo-oligosaccharides). The inventors observed that the relative proportion of each fraction was dependent on the severity index of the steam explosion pretreatment. In particular, the inventors found that, as illustrated in FIG. 12, the proportion of each fraction varied non-linearly with the severity index. In particular, the degradation of hemicellulose into inhibitory volatiles accelerated above a severity index of about 3.7, the conversion of the solids fraction into solubles and volatiles slowed down above a severity index of about 4, the proportion of the recoverable solubles decreased above a severity of about 4 and the proportion of the monomeric portion derived from the starting solids remained static above a severity index of about 4. Thus, the inventors found that by choosing a severity index of about 4 for the autohydrolysis of the biomass, three different optimization goals that were previously not considered in combination can be achieved simultaneously in relation to the hydrolysis of the hemicellulose fraction of the biomass. The inventors discovered that relative maximization of the hemicellulose solids breakdown, maximization of the proportion of xylo-oligosaccharides generated, and relative minimization of the hemicellulose degradation can all be achieved simultaneously if the hemicellulose fraction of the biomass is subjected to autohydrolysis by steam pretreatment at a severity index of about 4.

Moreover, the inventors found that the individual hemicellulose fractions generated during pretreatment can be captured separately during pretreatment, rather than separated in downstream steps. In particular, the inventors discovered that a vapor phase containing mostly inhibitory volatiles can be captured by purging the volatiles during pretreatment as well as right after explosive decompression. Volatiles recovered by purging included a high proportion of degradation products not desirable in purified xylo-oligosaccharides and inhibitory to cellulose hydrolysis and/or fermentation. The solubles fraction of the hydrolyzed hemicellulose can be captured separately by purging condensate during pretreatment as well as right after explosive decompression, liquid extracting the solids fraction during pretreatment after purging of the condensate and combining the purge streams and the liquid extracted solubles into a combined solubles stream including mainly xylose monomers and oligomers as well as a minor portion of degradation products. The inventors have found that by separately capturing the volatiles, the condensate and the liquid extracted solubles, the majority of degradation compounds created during autohydrolysis of the biomass is separately captured in the vapor stream with volatiles, while only a minor portion of degradation products is still found in the combined solubles stream, making it possible to render the combined solubles stream into a purified, xylo-oligosaccharides rich liquid fraction by a cost efficient evaporation step for driving out the remaining degradation products.

On the basis of this research, the inventors have developed a continuous pretreatment process incorporating the findings relating to the influence of the severity index of the autohydrolysis pretreatment on the composition of the hemicellulose hydrolysis products mix with the findings relating to the fractionation of the hemicellulose hydrolysis products during pretreatment. This pretreatment process allows for the continuous fractionation of lignocellulosic biomass into a cellulose-rich fraction, a xylo-oligosaccharides-rich fraction and a vapor fraction containing inhibitors. This is achieved by first obtaining biomass, or a mixture of biomasses, having a lignin content of less than 12% and an acetyl group content of 3-6% and then subjecting this biomass to steam explosion pretreatment at a severity index of about 4 to maximize autohydrolyis of the hemicellulose fraction in the biomass and maximize a proportion of xylo-oligosaccharides generated, while minimizing hemicellulose degradation. During the steam pretreatment exposure time, liquid condensate, cooking liquids and vapor generated during autohydrolysis are captured in a first liquid stream with hemicellulose sugars free of lignin and water soluble compounds, and a first vapor stream with volatile chemicals. After the autohydrolysis step, the prehydrolyzed biomass still under pressure is liquid extracted to obtain a liquid stream containing hemicellulose sugars free of lignin and hemicellulose degradation components. After this liquid extraction step, the reaction pressure is rapidly released to afford explosive decompression of the extracted, prehydrolyzed lignocellulosic biomass into fibrous solids, vapor with hemicellulose degradation components and volatile chemicals inhibitory to fermentation, as well as condensate containing mostly hemicellulose sugars free of lignin. Vapor and condensate generated during the explosive decompression are separately captured as a second vapor stream and a second liquid stream. The first and second liquid streams are then combined with the liquid hemicellulose degradation stream into a liquids fraction which is subsequently subjected to evaporation of the hemicellulose degradation products for separation and recovery of a xylo-oligosaccharides rich solution.

The preferred process of the invention includes the steps of obtaining lignocellulosic biomass having a content of less than 12% lignin by weight in the dry matter and an acetyl group content of 3-6% by weight in the dry matter, exposing the optionally ground, lignocellulosic biomass to steam at 170° C. to 220° C. at 100 to 322 psig for 2 to 300 minutes without the use of mineral acid catalysts. The term lignocellulosic biomass in this context is meant to cover both a specific biomass as well as a mixture of biomasses, as long as the lignin and acetyl group content are as required. The pretreatment preferably includes the continuous purging of volatile and liquid compounds. The exposing step preferably steam treats the biomass to a temperature and retention time with translates into a Severity Index of 3.9 to 4.1, most preferably about 4, the Severity Index being calculated according to the equation:

Severity Index=Log×Exp[(Temperature ° C.−100)/14.75]×Retention Time(min).

Steam pretreating corncobs at a severity index of 4 leads to a final pH of 3.5 to 4.0 of the pretreated biomass, while the same severity index leads to a final pH of 4.5 to 5 with bagasse biomass.

The process also includes liquid extraction of the steam pretreated fibres under pressure with/or without eluent addition to remove water soluble hemicelluloses, acids and hemicellulose and cellulose breakdown and degradation products. As an option, these compounds, which are inhibitors of downstream hydrolysis and fermentation may be extracted during pretreatment, after pretreatment, or both during and after pretreatment. The extraction of the soluble compounds from the pretreated fibers preferably results in 4% to 10% xylose based sugars consisting of polymers (xylan), monomers and xylo-oligosaccharides remaining in the prehydrolysis fibers.

The extracted fibers, also referred to as prehydrolysate, are then separated from the gaseous reaction products in a cyclone separator and collected at the bottom of the separator, shredded and diluted to a desired consistency and subsequently transported to the enzymatic hydrolysis step.

The collected prehydrolysate is then shredded, diluted with water to 10-30% consistency and then reacted with cellulase enzymes to produce glucose. The glucose rich solution is readily utilized in the subsequent fermentation step where an organism converts the glucose into ethanol.

### Example 1

### Autohydrolysis Pretreatment Process

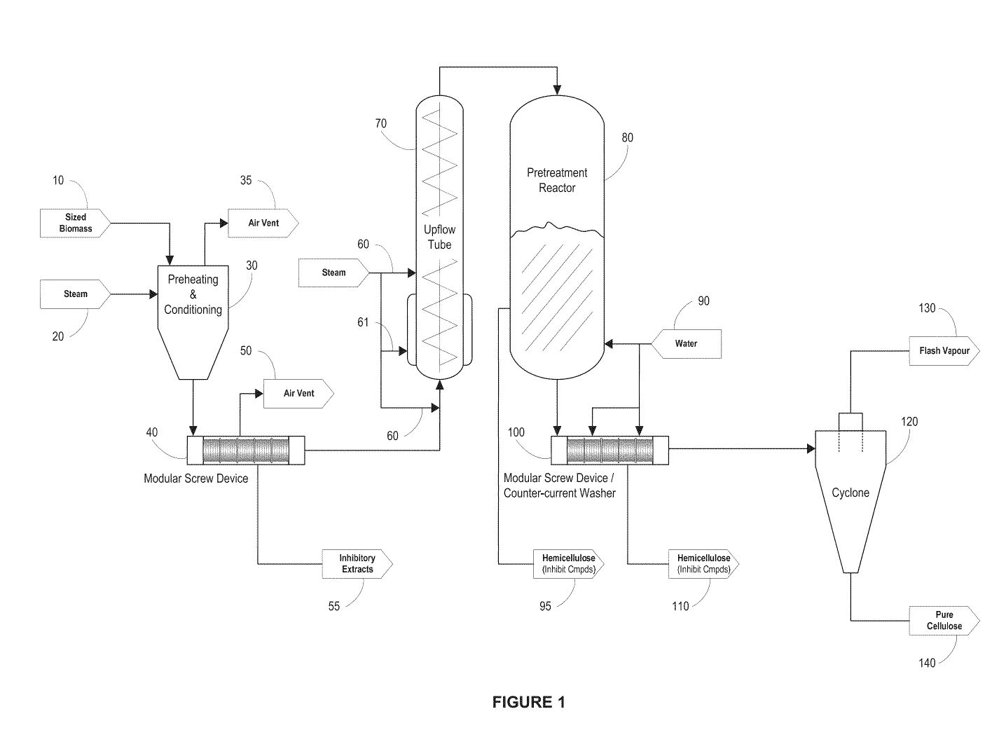

In the following example, reference numbers refer to features of the pretreatment system and process streams, as shown in FIG. 1.

Continuous steam explosion pretreatment of lignocellulosic biomass is carried out in a steam explosion pretreatment system. In this example the biomass is corncobs.

Corncobs 10 are received, stored, cleaned, ground (0.5 to 1 cm3 particle size) and fed through a V shaped hopper and screw auger (not shown). The corncob moisture is adjusted to 50% DM.

Prepared corncobs are pre-conditioned by preheating them with live steam 20 at atmospheric pressure, in a holding bin or preheating and conditioning container 30 to a temperature of about 95° C. for about 10-60 minutes. Air and steam are vented through an air vent 35 from the preheating and conditioning container 30.

Preheated corncobs are compressed in a first modular screw to remove air 50 through an air vent and inhibitory extracts 5. The corncobs are then fed into a pressurized upflow tube 70.

Pressurized saturated steam at a temperature of 205° C. is injected upstream of and/or directly into the upflow tube 70 by direct injection 60 and/or indirect injection of steam 61 in a jacketed section of the upflow tube until the desired cooking pressure is reached.

Corncobs are moved through the upflow tube with the aid of a screw conveyor/mixer (3 min) and are discharged into a pretreatment reactor 80.

Corncobs are continuously discharged from the pretreatment reactor 80 to a second pressurized modular screw device 100 after a residence time of 5 min at 205° C. in the pretreatment reactor 80. This results in a treatment severity index of 4).

During the residence time, condensate and cooking liquids collected at the bottom of the pretreatment reactor are purged through a purge discharge control valve 95.

Pretreated corncobs are washed with water eluent under pretreatment pressure. Hot water 90 is added to dilute the pretreated corncobs as the fiber is discharged from the pretreatment reactor. Further hot water is also added along the pressing device 100 to reach a ratio of about 6:1 wash water:corncobs and to achieve a greater extraction of hemicellulose. The extracted hemicellulose solution 110 is collected and concentrated to the desired dryness for further applications.

The pressurized washed corncobs are then flashed into a cyclone 120. The solids, i.e. purified cellulose, collected at the bottom of the cyclone separator and are subjected to further processing i.e. shredded and then diluted with fresh water to the desired consistency for hydrolysis and fermentation.

The gaseous components are collected, condensed and fed to a condensate tank 130. Any gaseous emissions from the pretreatment reactor, the cyclone separator and other parts of the steam gun setup are collected and treated in an environmental control unit (not shown). Cleaned gases are exhausted to atmosphere from the environmental control unit.

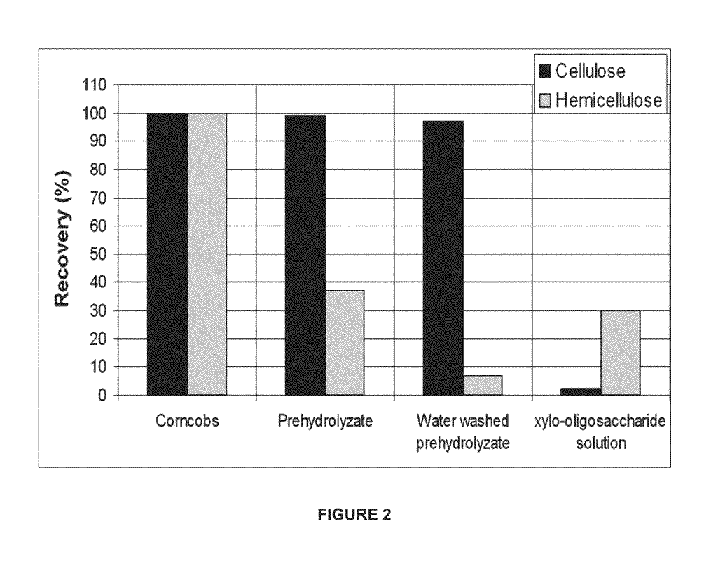

In order to simulate this new process, steam explosion pretreatment of corncobs was followed by batch washing at pilot scale with a 97% recovery of cellulose (FIG. 2).

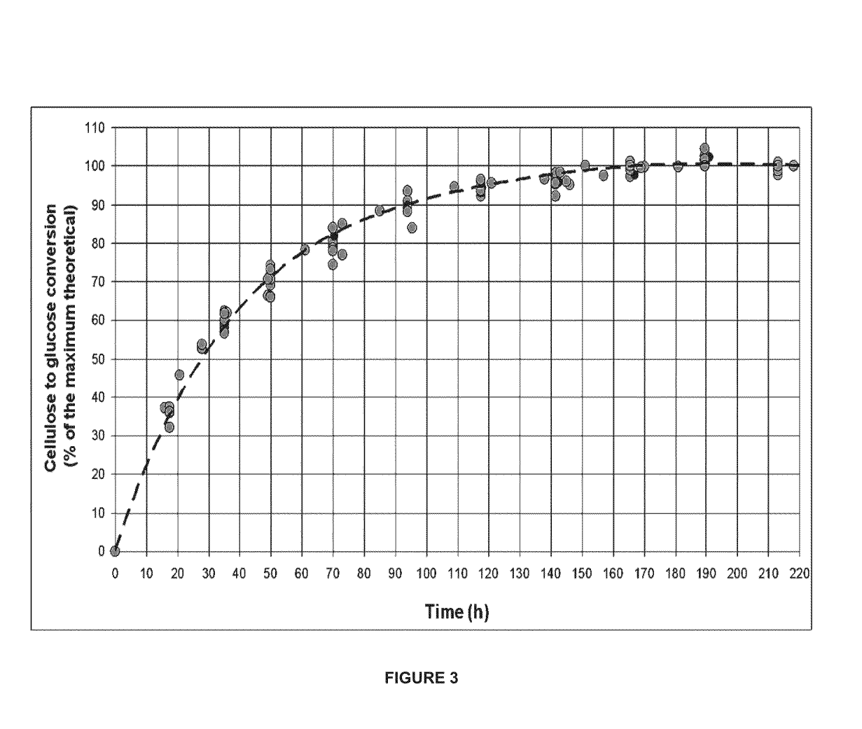

Extracted cellulose from the pilot scale pretreatment was highly susceptible to enzymatic hydrolysis. 80% of the maximum theoretical cellulose to glucose conversion was achieved in 60 h. 90% conversion of the 17% consistency slurry was reached in 95 h, using only 0.23% load of commercial cellulases product (FIG. 3).

In FIG. 3, the dashed line represents the trend of eleven enzymatic hydrolysis experiments carried out at three different scales (i.e. 1 kg, 300 kg and 2500 kg). These enzymatic hydrolysis experiments were carried out at 17% consistency, 50° C. and pH 5.0.

The pH adjustment chemical used was aqueous ammonia (30%). Commercially available lignocellulolytic enzyme was used at a load of 0.23% weight/weight on incoming cob feedstock.

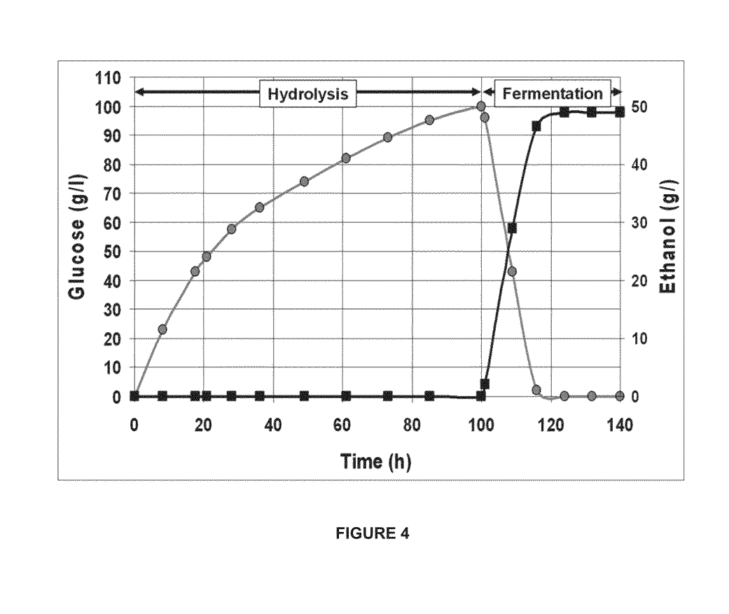

Samples of the continuously pretreated corncobs were hydrolyzed and fermented in a 2.5 metric tonne batch hydrolysis and fermentation trial (FIG. 4). The results were in accordance with the lower scale pilot and the laboratory scale results (FIG. 3). A concentration of 100 g/L glucose was reached at t 90% i.e. 100 hours hydrolysis of 17% consistency slurry, using only 0.23% load of commercial cellulase product.

The fermentability of the hydrolyzed cellulose was high. A concentration of 4.9% alcohol was reached in 20 hours (FIG. 4).

In FIG. 4, hydrolysis was carried out at 50° C., pH 5.0 and 0.23% enzyme load. Fermentation was carried out at 33° C., pH 5.3, using industrial-grade C6-fermenting yeast. Hydrolysis and fermentation pH adjustment was carried out using aqueous ammonia (30%). Grey circles indicate glucose concentration. Black squares indicate ethanol concentration.

The production of soluble xylo-oligosaccharides was equivalent to 12% of the weight of raw corncobs processed at pilot scale. 63% of the original content of corncobs hemicellulose was converted to volatile degradation products (FIG. 2). 66% of these volatiles were flashed off during the step of explosive decompression.

81% of the hemicellulose remaining in the corncobs prehydrolysate after autohydrolysis was collected through the prehydrolysate water washing step. The resulting lignin free solution contained dissolved solids of which 87% were sugars, including 63% of xylo-oligosaccharides (w/w) on a dry matter basis.

### Example 2

### High Pressure Pretreatment of Corncobs

Steam explosion pretreatment of corncobs was carried out in a steam explosion pretreatment system pressurized with saturated steam at a temperature of 205° C. No acid was added to the corncobs during the heating step. The corncob moisture was adjusted to 60% DM. The overall retention time of corncob pretreatment was 8 min e.g. 3 min in the up flow tube, 5 min in the pretreatment reactor at pH 3.8. Corncob acidification resulted from the release of acetic acid from hemicellulose breakdown.

Pretreated corncobs were water washed.

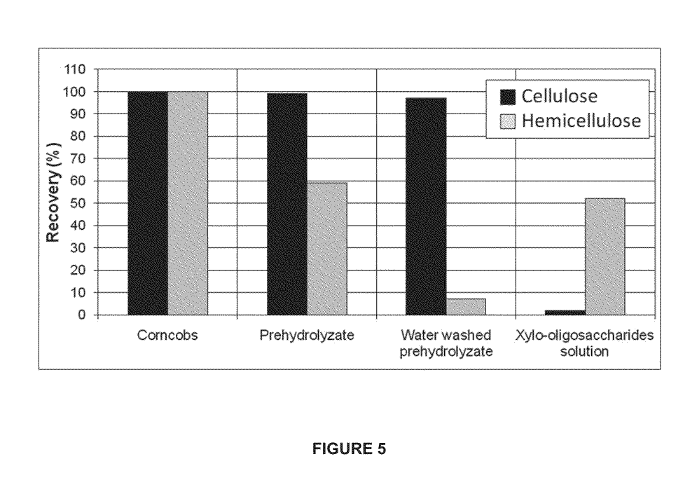

Cellulose extraction from corncobs was carried out at pilot scale with a percentage recovery of 98% (FIG. 5).

59% of the incoming hemicellulose was recovered after high pressure pretreatment of corncobs. 52% of incoming hemicellulose was collected into the xylo-oligosaccharides solution (FIG. 5). The resulting lignin free solution contained 89% sugars, including 66% of xylo-oligosaccharides (w/w) on a dry matter basis.

### Example 3

### Low Pressure Pretreatment of Corncobs

Steam explosion pretreatment of corncobs was carried out in a steam explosion pretreatment system pressurized with saturated steam at a temperature of 170° C. No acid was added to the corncobs during the heating step. The corncob moisture was adjusted to 50% DM. The overall retention time of corncobs pretreatment was 85 min e.g. 15 min in an up flow tube, 70 min in a pretreatment reactor at pH 3.8. Corncob acidification resulted from the release of acetic acid from hemicellulose breakdown.

Pretreated corncobs were water washed.

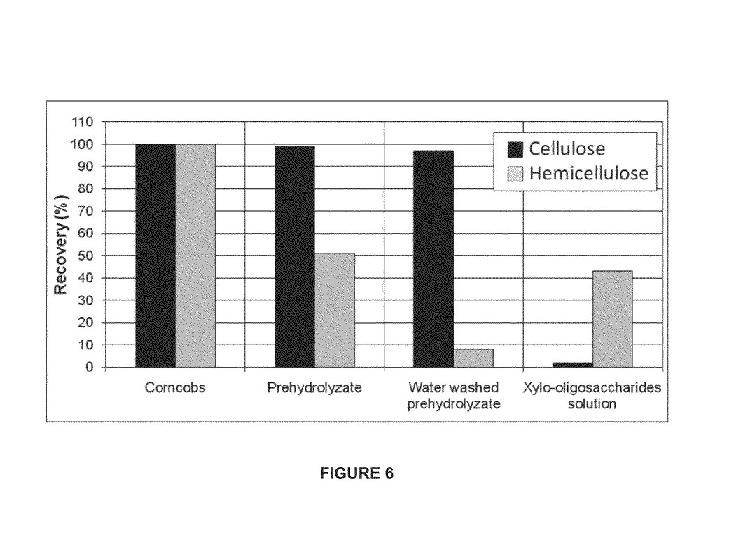

Cellulose extraction from corncobs was carried out at pilot scale with a percentage recovery of 98% (FIG. 6).

51% of incoming hemicellulose was recovered after low pressure pretreatment of corncobs. 43% of incoming hemicellulose was collected in the xylo-oligosaccharides solution (FIG. 6). The resulting lignin free solution contained 88% sugars, including 65% of xylo-oligosaccharides (w/w) on a dry matter basis.

After explosive decompression, the solid fraction from high or low pressure pretreatment i.e. purified cellulose was collected at the bottom of cyclone separator, shredded and then diluted with fresh water up to 17% consistency.

Extracted cellulose from high and low pressure continuous pilot scale pretreatment of corncobs was highly susceptible to enzymatic hydrolysis. Digestibility of cellulose pretreated at high and low pressure was similar (FIG. 3). 80% of the maximum theoretical cellulose to glucose conversion was achieved in 60 h. 90% conversion of the 17% consistency slurry was reached in 95 h, using only 0.23% load of commercial cellulases product (FIG. 3).

In FIG. 3, the dashed line represents the trend of six duplicate enzymatic hydrolysis experiments carried out at three different scales (i.e. 1 kg, 300 kg and 2500 kg) with cellulose extracted at high or low pressure. These enzymatic hydrolysis experiments were carried out at 17% consistency, 50° C. and pH 5.0. The pH adjustment chemical used was aqueous ammonia (30%). Commercially available lignocellulolytic enzyme was used at a load of 0.23% weight/weight on incoming cob feedstock.

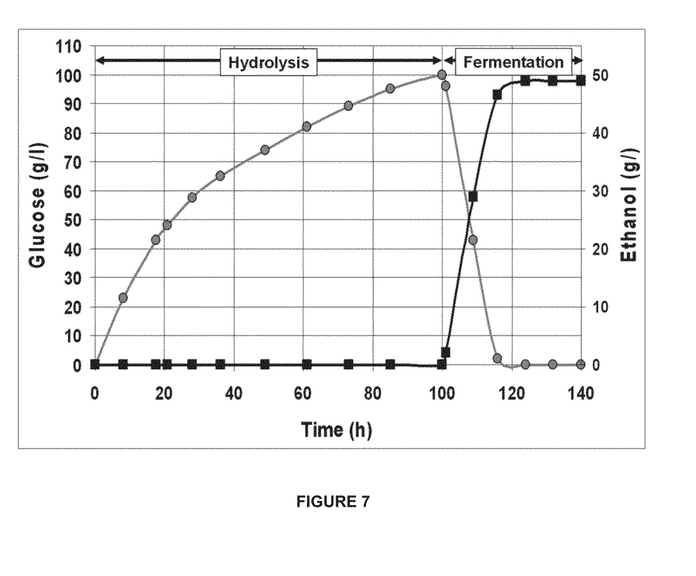

At pilot scale (2.5 metric tonne fed batch hydrolysis and fermentation trial, FIG. 7) a concentration of 100 g/L glucose representing 91% conversion of the cellulose was reached after 100 hours hydrolysis of a 17% consistency slurry from low pressure pretreatment.

In FIG. 7, hydrolysis was carried out at 50° C., pH 5.0 and 0.23% enzyme load. Fermentation was carried out at 33° C., pH 5.3, using industrial grade C6-fermenting yeast. Hydrolysis and fermentation pH adjustment was carried out using aqueous ammonia (30%). Grey circles indicate glucose concentration. Black squares indicate ethanol concentration.

Fermentability of the hydrolyzed cellulose was evaluated by adding enough C6-industrial grade commercial yeast to reach a concentration of 10⁸ yeast cells per gram hydrolysate at 33° C., pH 5.3 when 90% of the maximum theoretical cellulose to glucose conversion was reached. pH adjustment was carried out with aqueous ammonia (30%) prior to yeast addition.

Fermentability of the hydrolyzed cellulose was high. A concentration of 4.9% alcohol was reached in 20 hours (FIG. 7).

### Example 4

### High Pressure Pretreatment of Bagasse

Steam explosion pretreatment of Bagasse was carried out in a system pressurized with saturated steam at a temperature of 205° C. No acid was added to the bagasse fibers during the heating step. The overall retention time of the bagasse fibers during pretreatment was 8 min e.g. 3 min in the up flow tube and 5 min in the pretreatment reactor at pH 4.8. Bagasse acidification resulted from the release of acetic acid from hemicellulose breakdown.

Pretreated Bagasse was water washed.

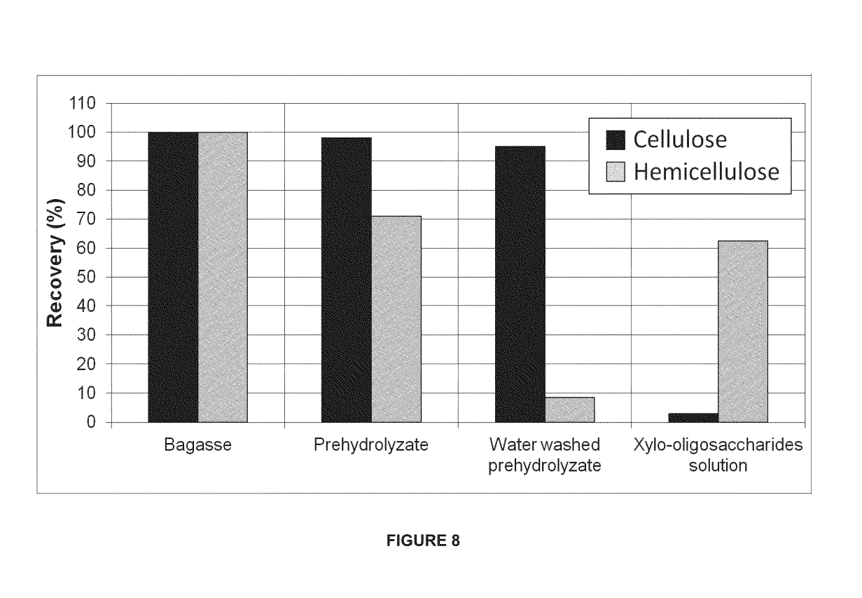

Cellulose extraction from the pretreated and washed Bagasse mash was carried out at pilot scale with a percentage recovery in the solid fraction of 95% (FIG. 8).

72% of the incoming hemicellulose was recovered after pretreatment of Bagasse. 63% of the incoming hemicellulose was collected in the xylo-oligosaccharides solution (FIG. 8). The resulting lignin free solution contained 85% sugars, including 62% of xylo-oligosaccharides (w/w) on a dry matter basis.

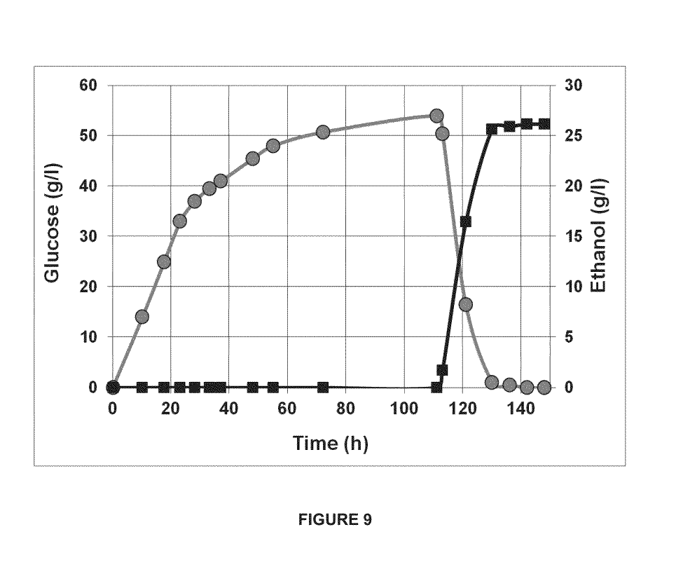

Extracted cellulose from pilot scale pretreatment of Bagasse was highly susceptible to enzymatic hydrolysis. 80% of the maximum theoretical cellulose to glucose conversion was achieved in 110 h (FIG. 9).

In FIG. 9, hydrolysis was carried out at 50° C., pH 5.0, using commercially available lignocellulolytic enzyme product at a load of 0.3% weight/weight on incoming cob feedstock. Fermentation was carried out at 33° C., pH 5.3 using an industrial-grade C6-fermnenting yeast.

A concentration of 54 g/L glucose representing 80% conversion of cellulose was reached after 110 hours of hydrolysis of a 13% consistency slurry, using only a 0.3% load of commercial cellulase.

Fermentability of the hydrolyzed cellulose was evaluated by adding enough C6-industrial grade commercial yeast to reach a concentration of 10⁸ yeast cells per gram hydrolysate at 33° C., pH 5.3. The time needed to reach the maximum theoretical cellulose to glucose conversion was determined. pH adjustment was carried out with aqueous ammonia (30%) prior to yeast addition.

The fermentability of the hydrolyzed cellulose was high. A concentration of 2.6% alcohol was reached in 20 hours (FIG. 9). This is equivalent to a glucose to ethanol conversion yield of 95%.

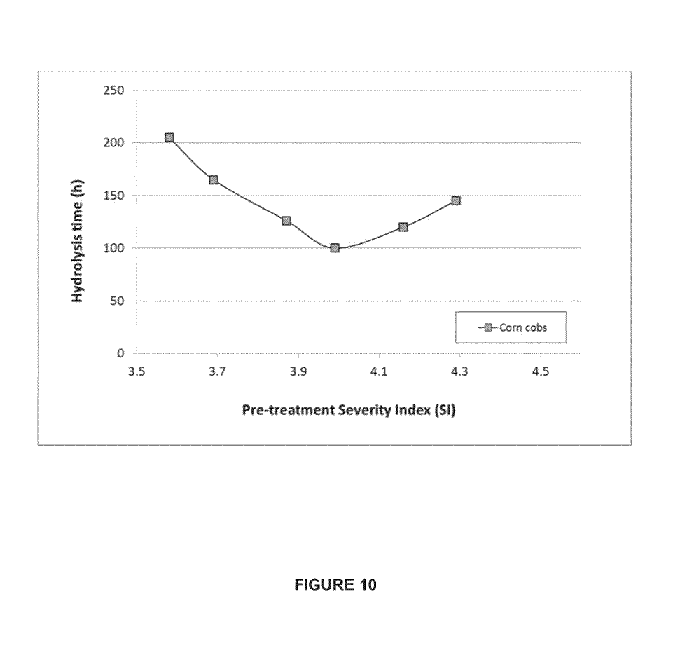

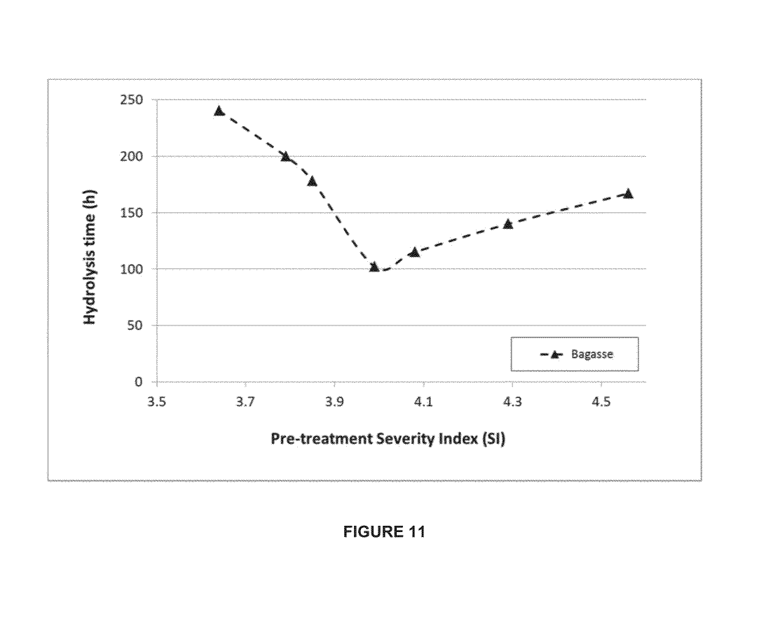

The achieved high degree of cellulose digestibility and cellulose to glucose conversion rates of cellulose derived from bagasse biomass subjected to pretreatment solely by autohydrolysis and without the addition of any acid catalyst was surprising. Numerous prior art references cited below teach the use of acid to improve hemicellulose hydrolysis during pretreatment for biomass having a low inherent acetic acid content. To date, it was not recognized in the art that due to the delicate interplay between the higher amount of hemicellulose breakdown achieved with added acid catalyst and the inhibitory effects of the breakdown products and the catalyst on the downstream cellulose hydrolysis and glucose fermentation processes, the use of acid catalyst for biomass with low acetic acid content is not always advantageous and may in fact lead to lower ethanol yields for certain lignocellulosic biomasses. The inventors have now surprisingly discovered that autohydrolysis without the addition of any acid catalyst can be carried out on lignocellulosic biomass of <12% lignin content and an acetyl group content of 3-6% weight/weight in the dry matter, with satisfactory ethanol yield and even higher ethanol yield compared to processes using added acid catalyst in the pretreatment step. In fact, as can be seen from FIGS. 10 and 11, optimal pretreatment conditions of corncob biomass and bagasse biomass with respect to the production of highly digestible cellulose and ethanol were found to be at exactly the same severity index and both without the addition of any acid catalyst. As is apparent from these Figures, the fastest time for digesting cellulose prehydrolysates was obtained with severity index of 4.0 SI in both cases, which is very surprising in view of the significant differences in lignin and acetyl group content of bagasse and corncob. The class of lignocellulosic biomasses of <12% lignin content and an acetyl group content of 3-6% weight/weight in the dry matter includes corncob, sugar cane bagasse, switchgrass, prairie grass, sorghum bagasse, corn stover, and wheat straw.

### REFERENCES

### Pretreatment of Low Lignocellulosic Biomass

(1) Shapouri H et al. (1995) USDA Report 721. Estimating the net energy balance of corn ethanol.
(2) Shapouri H et al. (2002) USDA Report 813. The Energy Balance of corn ethanol: an update.
(3) Chow J et al. (2003) Science, 302, 1528-1531 Energy resources and global development.
(4) Wald M L, Barrionuevo A (2007) New York Times, April 7th, The Energy challenge: A Renewed push for ethanol, without the corn.
(5) Greeg D (2008) Biocycle, 49, 11-47. Commercializing cellulosic ethanol.
(6) Hill J et al. (2006) Proc. Natl. Acad. Sci. USA, 103, 11206-11210. Environmental, economic, and energetic costs and benefits of biodiesel and ethanol biofuels.
(7) Farrell A E et al. (2006) Science, 311, 506-508. Ethanol can contribute to energy and environmental goals.
(8) Somerville C (2007) Current biology, 17, 115-119. Biofuels.
(9) Schuetzle D et al. (2007) Western Governors' Association. Alcohol fuels from biomass-Assessment of production technologies.
(10) Chum L, Overend R (2002) Fuel Processing technology, 71, 187-195. Biomass and renewable fuels.
(11) Wyman C E (1996) Taylor & Francis: Washington D.C., USA, Handbook on bioethanol: production and utilization.
(12) Delmer D P, Amor Y (1995) Plant Cell, 7, 987-1000. Cellulose biosynthesis.
(13) Morohoshi N (1991) In Wood and cellulosic chemistry; Hon, D. N. S, Shiraishi, N., Eds.; Marcel Dekker, Inc.: New York, USA, Chemical characterization of wood and its components.
(14) Ha M A et al. (1998) Plant J. 1998, 16, 183-190. Fine structure in cellulose microfibrils: NMR evidence from onion and quince.
(15) Palmqvist E, Hahn-Hägerdal B (2000) Bioresource Technol., 74, 25-33. Fermentation of lignocellulosic hydrolysates. II: Inhibitors and mechanisms of inhibition.
(16) De Vrije T et al (2002) International journal of hydrogen energy, 27, 1381-1390. Pretreatment of miscanthus for hydrogen production by thermotoga elfii.
(17) Galbe M, Zacchi G (2002) Appl Microbiol Biotechnol 59 618-628. A review of the production of ethanol from softwood.
(18) Torget R et al. (1991) Bioresource Technol., 35, 239-246. Dilute sulfuric acid pretreatment of hardwood bark.
(19) Donghai S et. al. (2006) Chinese J. Chem. Eng., 14, 796-801. Effects of different pretreatment modes on the enzymatic digestibility of corn leaf and corn stalk.
(20) Renewable Fuels for advanced Powertrains-Final report (2008) www.renew-fuel.com/download.php?dl=renew-final-report-080627.pdf&kat=5
(21) Bullard M, Metcalfe P (2001) Crown publisher ETSU B/U1/00645/REP DTI/Pub URN 01/797. Estimating the energy requirement and CO2 emission from production of the perennial grasses miscanthus, switchgears and reed canary grass.
(22) Newman R (2003) Crown publisher ETSU B/W2/00618/REP-URN 03/1568. Miscanthus-Practical aspects of biofuel development.
(23) Christian D G, Haase E (2001). Agronomy of Miscanthus. In M. B. Jones & M. Walsh, Miscanthus for energy and fibre. London: James and James.
(24) Planting and Growing Miscanthus (2007) Department for Environment, Food and Rural Affairs (Defra) http://www.defra.gov.uk/erdp/pdfs/ecs/miscanthus-guide.pdf
(25) Papatheofanous M G et al. (1995) Biomass and Bioenergy, 8, 419-426. Biorefining of agricultural crops and residues: effect of pilot-plant fractionation on properties of fibrous fractions.
(26) Lange W (1992) Holzforschung, 46, 277-282. Extracts of miscanthus grass (Miscanthus sinensis Anderss.). A comparison of the ‘summer-green’ and the ‘winter-dry’ plant.
(27) Sims, R (2003) Elsevier Science: London, UK, Biomass and resources bioenergy options for a cleaner environment in developed and developing countries.
(28) Sjöström, E (1993) Academic Press: San Diego, USA, Wood chemistry: fundamentals and applications.
(29) Price L et al. (2004) Biomass and Bioenergy, 3-13. Identifying the yield potential of Miscanthus×giganteus: an assessment of the spatial and temporal variability of M.×giganteus biomass productivity across England and Wales.
(30) Velasquez J A et al. (2003) Wood Science and Technology, 37, 269-278. Binderless fiberboards from steam exploded Miscanthus sinensis.
(31) Lewandowski I et al. (2003) Agronomy J, 95, 1274-1280. Environment and harvest time affects the combustion qualities of Miscanthus genotypes.
(32) Kurakake M et al (2001) Applied biochemistry and Biotechnology, 90, 251-259. Pretreatment with ammonia water for enzymatic hydrolysis of corn husk, bagasse, and switchgrass.
(33) Lewandowski I et al. (2003) Biomass and Bioenergy, 25, 335-361. Development and current status of perennial rhizomatous grasses as energy crops in the US and Europe.
(34) Sun Y, Cheng J (2002) Bioresources Technol., 83, 1-11. Hydrolysis of lignocellulosic materials for ethanol production: A review.
(35) McMillan J D (1994) In Enzymatic Conversion of Biomass for Fuels Production; Himmel, M. E., Baker, J. O., Overend, R. P., Eds.; ACS: Washington D.C., USA, 1994; pp. 292-324. Pretreatment of lignocellulosic biomass.
(36) Fan L et al (1982) Adv. Biochem. Eng. Biotechnol., 23, 158-183. The nature of lignocellulosics and their pretreatments for enzymatic hydrolysis.
(37) Mosier N et al. (2005) Bioresources Technol, 96, 673-686. Features of promising technologies for pretreatment of lignocellulosic biomass.
(38) Henley R G et al. (1980) Enzyme Microb. Tech., 2, 206-208. Enzymatic saccharification of cellulose in membrane reactors.
(39) Berlin A et al. (2006) J. Biotechnol., 125, 198-209. Inhibition of cellulase, xylanase and beta-glucosidase activities by softwood lignin preparations.
(40) Chandra R et al. (2007) Adv. Biochem. Eng. Biotechnol, 108, 67-93. Substrate pretreatment: The key to effective enzymatic hydrolysis of lignocellulosics?
(41) Kassim E A, El-Shahed A S (1986) Agr. Wastes, 17, 229-233. Enzymatic and chemical hydrolysis of certain cellulosic materials.
(42) Xu Z et al (2007) Biomass Bioenerg. 2007, 31, 162-167. Enzymatic hydrolysis of pretreated soybean straw.
(43) Vaccarino C et al (1987) Biol. Waste, 20, 79-88. Effect of SO2NaOH and Na2CO3 pretreatments on the degradability and cellulase digestibility of grape marc.
(44) Silverstein R A et al (2007) Bioresource Technol., 2007, 98, 3000-3011. A comparison of chemical pretreatment methods for improving saccharification of cotton stalks.
(45) Zhao X et al (2007) Bioresource Technol., 99, 3729-3736. Comparative study on chemical pretreatment methods for improving enzymatic digestibility of crofton weed stem.
(46) Gaspar M et al (2007) Process Biochem., 2007, 42, 1135-1139. Corn fiber as a raw material for hemicellulose and ethanol production.
(47) Saha B C, Cotta M A (2006) Biotechnol. Progr., 22, 449-453. Ethanol production from alkaline peroxide pretreated enzymatically saccharified wheat straw.
(48) Saha B C, Cotta M A (2007) Enzyme Microb. Tech., 41, 528-532. Enzymatic saccharification and fermentation of alkaline peroxide pretreated rice hulls to ethanol.
(49) Mishima D et al (2006) Bioresource Technol. 2006, 97, 2166-2172. Comparative study on chemical pretreatments to accelerate enzymatic hydrolysis of aquatic macrophyte biomass used in water purification processes.
(50) Sun X F et al (2005) Carbohyd. Res., 340, 97-106. Characteristics of degraded cellulose obtained from steam-exploded wheat straw.
(51) Alizadeh H et al (2005) Appl. Biochem. Biotechnol., 124, 1133-41. Pretreatment of switchgrass by ammonia fiber explosion (AFEX).
(52) Chundawat S P et al (2007) Biotechnol. Bioeng., 96, 219-231. Effect of particle size based separation of milled corn stover on AFEX pretreatment and enzymatic digestibility.
(53) Eggeman T, Elander R T. (2005) Bioresource Technol., 96, 2019-2025. Process and economic analysis of pretreatment technologies.
(54) Chum H L (1985) Solar Energy Research Institute: Golden, Colo., 1-64. Evaluation of pretreatments of biomass for enzymatic hydrolysis of cellulose.
(55) Taherzadeh M J, Karimi K (2007) Bioressources, 2, 472-499. Process for ethanol from lignocellulosic materials I: Acid-based hydrolysis processes.
(56) Ruiz E et al (2008) Enzyme Microb. Tech., 42, 160-166. Evaluation of steam explosion pretreatment for enzymatic hydrolysis of sunflower stalks.
(57) Ballesteros M et al. (2004) Process Biochem., 39, 1843-1848. Ethanol from lignocellulosic materials by a simultaneous saccharification and fermentation process (SFS) with Kluyveromyces marxianus CECT 10875.
(58) Negro M J et al (2003) Appl. Biochem. Biotechnol., 105, 87-100. Hydrothermal pretreatment conditions to enhance ethanol production from poplar biomass.
(59) Kurabi A et al (2005) Appl. Biochem. Biotechnol., 121-124. Enzymatic hydrolysis of steam exploded and ethanol organosolv-pretreated Douglas-firby novel and commercial fungal cellulases.
(60) Varga E et al (2004) Appl. Biochem. Biotechnol., 509-523. Optimization of steam pretreatment of corn stover to enhance enzymatic digestibility.
(61) Eklund R (1995) Bioresource Technol., 52, 225-229. The influence of SO2 and H2SO4 impregnation of willow prior to steam pretreatment.
(62) Yang B, Wyman C E (2004) Biotechnol. Bioeng, 86, 88-95. Effect of xylan and lignin removal by batch and flowthrough pretreatment on the enzymatic digestibility of corn stover cellulose.
(63) Eggeman T, Elander R T. (2005) Bioresource Technol., 96, 2019-2025. Process and economic analysis of pretreatment technologies.
(64) Girio F M F (1997) FAIR-CT97-3811. Development of xylooligosaccharides and xylitol for use in pharmaceutical and food industries (Xylophone)
(65) Izumi et al (2002) US patent application No. 2002195213 A1. Process for producing xylooligosaccharide from lignocellulose pulp., 16pp
(66) Yu S et al (2002) Faming Zhuanli Shengqing Gongkai Shuomingshu, CN 1364911 A, 7pp Preparation of xylooligosaccharide by degradation of plant fiber with enzyme.
(67) Kabel M A et al (2002) Carbohydrate Polymers, 50, 47-56. Hydrothermally treated xylan rich by-products yield different classes of xylo-oligosaccharides.
(68) Werpy T, Petersen G (2004) DOE—Top Value Added Chemicals from Biomass—Volume I: Results of screening for potential candidates from sugars and synthesis gas http://www1.eere.energy.gov/biomass/pdfs/35523.pdf
(69) Carvalheiro F et al (2008) Journal of Scientific & Industrial Research, 67, 849-864. Hemicellulsoe biorefineries: a review on biomass pretreatments
(70) Ebringerova A (2006) Macromolecular Symposia, 232, 1-12. Structural diversity and application potential of hemicelluloses.

### REFERENCES

### Pretreatment of Corncobs

(71) Shapouri H et al. (1995) USDA Report 721. Estimating the net energy balance of corn ethanol.
(72) Shapouri H et al. (2002) USDA Report 813. The Energy Balance of corn ethanol: an update.
(73) Chow J et al. (2003) Science, 302, 1528-1531 Energy resources and global development.
(74) Wald M L, Barrionuevo A (2007) New York Times, April 7th, The Energy challenge: A Renewed push for ethanol, without the corn.
(75) Gregg D (2008) Biocycle, 49, 11-47. Commercializing cellulosic ethanol.
(76) Hill J et al. (2006) Proc. Natl. Acad. Sci. USA, 103, 11206-11210. Environmental, economic, and energetic costs and benefits of biodiesel and ethanol biofuels.
(77) Farrell A E et al. (2006) Science, 311, 506-508. Ethanol can contribute to energy and environmental goals.
(78) Somerville C (2007) Current biology, 17, 115-119. Biofuels.
(79) Schuetzle D et al. (2007) Western Governors' Association. Alcohol fuels from biomass-Assessment of production technologies.
(80) Chum L, Overend R (2002) Fuel Processing technology, 71, 187-195. Biomass and renewable fuels.
(81) Wyman C E (1996) Taylor & Francis: Washington D.C., USA, Handbook on bioethanol: production and utilization.
(82) Delmer D P, Amor Y (1995) Plant Cell, 7, 987-1000. Cellulose biosynthesis.
(83) Morohoshi N (1991) In Wood and cellulosic chemistry; Hon, D. N. S, Shiraishi, N., Eds.; Marcel Dekker, Inc.: New York, USA, Chemical characterization of wood and its components.
(84) Ha M A et al. (1998) Plant J. 1998, 16, 183-190. Fine structure in cellulose microfibrils: NMR evidence from onion and quince.
(85) Palmqvist E, Hahn-Hägerdal B (2000) Bioresource Technol., 74, 25-33. (84) Fermentation of lignocellulosic hydrolysates. II: Inhibitors and mechanisms of inhibition.
(86) De Vrije T et al (2002) International journal of hydrogen energy, 27, 1381-1390. Pretreatment of miscanthus for hydrogen production by thermotoga elfii.
(87) Galbe M, Zacchi G (2002) Appl Microbiol Biotechnol 59 618-628. A review of the production of ethanol from softwood.
(88) Torget R et al. (1991) Bioresource Technol., 35, 239-246. Dilute sulfuric acid pretreatment of hardwood bark.
(89) Donghai S et. al. (2006) Chinese J. Chem. Eng., 14, 796-801. Effects of different pretreatment modes on the enzymatic digestibility of corn leaf and corn stalk.
(90) Sun Y, Cheng J (2002) Bioresources Technol., 83, 1-11. Hydrolysis of lignocellulosic materials for ethanol production: A review.
(91) McMillan J D (1994) In Enzymatic Conversion of Biomass for Fuels Production; Himmel, M. E., Baker, J. O., Overend, R. P., Eds.; ACS: Washington D.C., USA, 1994; pp. 292-324. Pretreatment of lignocellulosic biomass.
(92) Fan L et al (1982) Adv. Biochem. Eng. Biotechnol., 23, 158-183. The nature of lignocellulosics and their pretreatments for enzymatic hydrolysis.
(93) Mosier N et al. (2005) Bioresources Technol, 96, 673-686. Features of promising technologies for pretreatment of lignocellulosic biomass.
(94) Henley R G et al. (1980) Enzyme Microb. Tech., 2, 206-208. Enzymatic saccharification of cellulose in membrane reactors.
(95) Berlin A et al. (2006) J. Biotechnol., 125, 198-209. Inhibition of cellulase, xylanase and beta-glucosidase activities by softwood lignin preparations.
(96) Chandra R et al. (2007) Adv. Biochem. Eng. Biotechnol, 108, 67-93. Substrate pretreatment: The key to effective enzymatic hydrolysis of lignocellulosics?
(97) Kassim E A, El-Shahed A S (1986) Agr. Wastes, 17, 229-233. Enzymatic and chemical hydrolysis of certain cellulosic materials.
(98) Xu Z et al (2007) Biomass Bioenerg. 2007, 31, 162-167. Enzymatic hydrolysis of pretreated soybean straw.
(99) Vaccarino C et al (1987) Biol. Waste, 20, 79-88. Effect of SO2NaOH and Na2CO3 pretreatments on the degradability and cellulase digestibility of grape marc.
(100) Silverstein R A et al (2007) Bioresource Technol., 2007, 98, 3000-3011. A comparison of chemical pretreatment methods for improving saccharification of cotton stalks.
(101) Zhao X et al (2007) Bioresource Technol., 99, 3729-3736. Comparative study on chemical pretreatment methods for improving enzymatic digestibility of crofton weed stem.
(102) Gaspar M et al (2007) Process Biochem., 2007, 42, 1135-1139. Corn fiber as a raw material for hemicellulose and ethanol production.
(103) Saha B C, Cotta M A (2006) Biotechnol. Progr., 22, 449-453. Ethanol production from alkaline peroxide pretreated enzymatically saccharified wheat straw.
(104) Saha B C, Cotta M A (2007) Enzyme Microb. Tech., 41, 528-532. Enzymatic saccharification and fermentation of alkaline peroxide pretreated rice hulls to ethanol.
(105) Mishima D et al (2006) Bioresource Technol. 2006, 97, 2166-2172. Comparative study on chemical pretreatments to accelerate enzymatic hydrolysis of aquatic macrophyte biomass used in water purification processes.
(106) Sun X F et al (2005) Carbohyd. Res., 340, 97-106. Characteristics of degraded cellulose obtained from steam-exploded wheat straw.
(107) Alizadeh H et al (2005) Appl. Biochem. Biotechnol., 124, 1133-41. Pretreatment of switchgrass by ammonia fiber explosion (AFEX).
(108) Chundawat S P et al (2007) Biotechnol. Bioeng., 96, 219-231. Effect of particle size based separation of milled corn stover on AFEX pretreatment and enzymatic digestibility.
(109) Eggeman T, Elander R T. (2005) Bioresource Technol., 96, 2019-2025. Process and economic analysis of pretreatment technologies.
(110) Chum H L (1985) Solar Energy Research Institute: Golden, Colo., 1-64. Evaluation of pretreatments of biomass for enzymatic hydrolysis of cellulose.
(111) Taherzadeh M J, Karimi K (2007) Bioressources, 2, 472-499. Process for ethanol from lignocellulosic materials I: Acid-based hydrolysis processes.
(112) Ruiz E et al (2008) Enzyme Microb. Tech., 42, 160-166. Evaluation of steam explosion pretreatment for enzymatic hydrolysis of sunflower stalks.
(113) Ballesteros M et al. (2004) Process Biochem., 39, 1843-1848. Ethanol from lignocellulosic materials by a simultaneous saccharification and fermentation process (SFS) with Kluyveromyces marxianus CECT 10875.
(114) Negro M J et al (2003) Appl. Biochem. Biotechnol., 105, 87-100. Hydrothermal pretreatment conditions to enhance ethanol production from poplar biomass.
(115) Kurabi A et al (2005) Appl. Biochem. Biotechnol., 121-124. Enzymatic hydrolysis of steam exploded and ethanol organosolv-pretreated Douglas-firby novel and commercial fungal cellulases.
(116) Varga E et al (2004) Appl. Biochem. Biotechnol., 509-523. Optimization of steam pretreatment of corn stover to enhance enzymatic digestibility.
(117) Eklund R (1995) Bioresource Technol., 52, 225-229. The influence of SO2 and H2SO4 impregnation of willow prior to steam pretreatment.
(118) Yang B, Wyman C E (2004) Biotechnol. Bioeng, 86, 88-95. Effect of xylan and lignin removal by batch and flowthrough pretreatment on the enzymatic digestibility of corn stover cellulose.
(119) Eggeman T, Elander R T. (2005) Bioresource Technol., 96, 2019-2025. Process and economic analysis of pretreatment technologies.
(120) Vazquez M, et al (2006) Industrial Crops and Products, 24, 152-159. Enhancing the potential of oligosaccharides from corncob autohydrolysis as prebiotic food ingredients
(121) Moura P, et al (2007) LWT, 40, 963-972. IN vitro fermentation of xylooligosaccharides from corncobs autohydrolysis by Bifidobacterium and Lactobacuillus strains.

What is claimed is:

1. A continuous process for fractionation of lignocellulosic biomass into a cellulose-rich solids fraction, a xylo-oligosaccharides-rich liquid fraction and a vapor fraction containing inhibitors to cellulose hydrolysis and/or fermentation, the method comprising the steps of:
a) obtaining biomass having a lignin content of less than 12% weight/weight in the dry matter and an acetyl group content of 3-6% weight/weight in the dry matter;
b) autohydrolysis of a hemicellulose fraction in the lignocellulosic biomass by exposing the biomass in a reaction vessel to steam at an elevated temperature and pretreatment pressure and for a preselected retention time to achieve a severity index of about 4 to maximize autohydrolysis of the hemicellulose fraction in the absence of any added acid catalyst, while simultaneously minimizing hemicellulose degradation, the severity index (SI) being calculated as
SI=Log×Exp[(treatment temperature(° C.)−100° C.)/14.75]×Retention Time(min),
to obtain a prehydrolyzed lignocellulosic biomass;
c) during the autohydrolysis step, purging liquid condensate, cooking liquids, and vapor generated by the autohydrolysis to remove and collect a first liquid stream with hemicellulose sugars free of lignin and water soluble compounds, and a first vapor stream with volatile inhibitors;
d) after the autohydrolysis step, liquid extracting under pretreatment pressure from the prehydrolyzed lignocellulosic biomass a liquid hemicellulose breakdown products stream containing hemicellulose sugars substantially free of lignin and hemicellulose degradation components;
e) after the liquid extracting step, rapidly releasing the reaction pressure to afford explosive decompression of the extracted, prehydrolyzed lignocellulosic biomass into a cellulose-rich fibrous solids fraction, vapor with hemicellulose degradation components and volatile chemicals inhibitory to fermentation, and a condensate containing mostly hemicellulose sugars free of lignin;
f) collecting the vapor released during the explosive decompression as a second vapor stream and the condensate formed during the explosive decompression as a second liquid stream;
g) combining the first and second liquid streams with the liquid hemicellulose breakdown products stream into a combined liquids stream; and
h) evaporating hemicellulose degradation products from the combined liquids stream for recovery of a xylo-oligosaccharides-rich liquid fraction.

2. A process for producing bio ethanol from lignocellulosic biomass having a lignin content of less than 12% and an acetyl group content of 3-6% weight/weight in the dry matter, comprising the steps of:
a) fractionating the biomass using the process of claim 1;
b) hydrolyzing the cellulose-rich fibrous solids fraction with cellulose enzymes to generate fermentable sugars;
d) adding ethanol producing yeast to the fermentable sugars to generate a fermentation broth; and
e) extracting ethanol from the fermentation broth.

3. The process of claim 1, where the lignocellulosic biomass is selected from the group consisting of corn cobs, miscanthus, sugar cane bagasse, switchgrass, prairie grass, sorghum bagasse, corn stover, and wheat straw.

4. The process of claim 1, wherein the process is carried out in a pretreatment exposing system and volatile compounds are removed continuously by venting the pretreatment exposing system.

5. The process of claim 1, wherein the process is carried out in a pretreatment exposing system and the purging of the liquid condensate takes place continuously at purging points in the pretreatment exposing system.

6. The process of claim 1, wherein an eluent is added to the pretreated and purged lignocellulosic biomass after the autohydrolysis step and prior to the step of extracting and removing the liquid hemicellulose breakdown products stream under pressure.

7. The process of claim 1, wherein the process is carried out in a pretreatment unit having a pretreatment reactor with an outlet connected to a solid-liquid separation device, and wherein wash water is added at a bottom of the pretreatment reactor and/or along the solid-liquid separation device to achieve a greater liquid extraction of the hemicellulose breakdown products stream.

8. The process of claim 1, wherein the solubilized by-products of hemicellulose breakdown created in the exposing step are extracted and removed from the solids fraction both before and after explosive decompression, with or without the addition of an eluent.

9. The process of claim 1, wherein extracted cellulose-rich solids fraction is separated from the liquid hemicellulose breakdown products stream by mechanical processing selected from the group consisting of compressing, filtering, centrifuging, and combinations thereof.
